(19) United States
(12) Patent Application Publication
(10) Pub. No.: US 2024/0041502 A1
(43) Pub. Date: Feb. 8, 2024

(54) SPINAL ROD COUPLER AND SYSTEM FOR SPINAL CORRECTIVE SURGERY

(71) Applicant: Johannes Hendrik Davis, Stellenbosch (ZA)

(72) Inventor: Johannes Hendrik Davis, Stellenbosch (ZA)

(21) Appl. No.: 18/267,093

(22) PCT Filed: Dec. 14, 2021
(86) PCT No.: PCT/ZA2021/050069

§ 371 Date: Jun. 13, 2023

(30) Foreign Application Priority Data
Dec. 14, 2020 (ZA) 2020/07771

(51) Int. Cl. A61B 17/70

(52) U.S. Cl. CPC A61B 17/7049; A61B 17/7043; A61B 17/7019

(57) ABSTRACT

This invention relates to a coupler (1) and more specifically, but not exclusively, to a coupler (1) for use in spinal corrective surgery. In accordance with the invention there is provided a coupler (1) comprising a first rod engaging member (2) and a second rod engaging member (3) which are attached to one another such that the rod engaging members (2, 3) may pivot relative to one another, to obtain a determined relationship and then progress to a fixed relationship to each other once definitively fastened. It is envisaged that the invention will provide a coupler (1) which has flexibility and multiple degrees of freedom available to allow manipulation, rotation and/or translation of individual vertebrae whereby allowing manipulations for corrective surgery which are bespoke to a patient.

### FIELD OF THE INVENTION

This invention relates to a spinal rod coupler and a system for spinal corrective surgery including a spinal rod coupler and more specifically, but not exclusively, to a spinal rod coupler and system for spinal corrective surgery for use with a single longitudinal rod and multiple transverse spinal rods.

### BACKGROUND TO THE INVENTION

A spinal rod is a metal implant use in spinal corrective surgery. Conventionally, where spinal rods are used to correct curvature of the spine, a pair of pre-contoured rods are affixed to the spine through pedicle screws. The pre contoured rods apply the necessary forces and moment to the spine to correct curvature defects or hold the bony elements firmly in place to allow bony healing after intervention that brings about fusion between spinal vertebrae. This conventional method has a number of disadvantages. Firstly, the pre-contoured rods need to be precisely shaped ahead of implantation to apply the corrective forces and moments. If, during surgery a surgeon realises that the pre-contoured rods are not adequately shaped to provide this, the rods have to be mechanically manipulated by hand during surgery, through in-situ bending with levers, that result in weak-points in the metal rods with risk for breakage or risk of screw pull-out, or the entire construct needs to be disassembled to allow further contouring of the rods prior to re-implantation. The pedicle screws may also be positioned such that it is difficult for the surgeon to engage the rod to the screw as desired.

Another problem is that, since the forces and moments act directly on the pedicle screws, there is a risk of screw pull-out as a result of forces which act longitudinally along the screws. This is especially true where vertebrae are rotated along a central axis of the spine, which will cause forces longitudinally on the screws, or where vertebrae need to be forcefully manipulated in a plane, in line with the longitudinal axis of the pedicle screws.

Another problem with the conventional method, encountered in scoliosis surgery using a pair of spinal rods, is altered shoulder balance, where one of the shoulders may be elevated (or in other words depressed in comparison to the other), as the shoulder girdle is attached to the spinal column and effective lengthening or shortening of the spinal column on one side in a transversal plane, might unbalance or tilt the shoulders and leave a cosmetically and functionally unappealing result.

### OBJECT OF THE INVENTION

It is accordingly an object of this invention to provide a spinal rod coupler and system for spinal corrective surgery which, at least partially, addresses or alleviates the disadvantages associated with the prior art or which provides a useful alternative to the prior art.

### SUMMARY OF THE INVENTION

In accordance with the invention there is provided a spinal rod coupler comprising:
a first rod engaging member and a second rod engaging member pivotably attached to each other;
the first engaging member being engagable to a first rod such that, when engaged, the member is movable longitudinally along the first rod and the first engaging member having first securing means for securing the engaging member to the first rod longitudinally along the first rod;
the second engaging member being engagable to a second rod such that, when engaged, the member is movable longitudinally along the second rod and the second engaging member having second securing means for securing the second rod engaging member to the second rod longitudinally along the second rod; and
pivot securing means for pivotally securing the first rod engaging member and second rod engaging member relative to each other;
wherein a position of the coupler and relative positions of the rods may be adjusted longitudinally along the rods and pivotally between the rods and secured in place by the securing means.

The coupler may include combined securing means wherein one or more of the first securing means, the second securing means, and the pivot securing means is actuated by a single mechanism.

The rod engaging members may be spinal rod engaging members.

The first rod engaging member is a transverse rod engaging member for engaging a transverse rod and the second rod engaging member is a longitudinal rod engaging member for engaging a longitudinal rod which extends crosswise, or at any angle, relative to the transverse rod.

Each engaging member may include a sleeve which slidably engages a rod. The sleeve may be an enclosed tubular structure or an open channel to accommodate the rod or member. The channel may be a U-shaped or C-shaped channel.

The sleeve may include securing means in the form of a fastener for fastening the sleeve to a rod. The fastener may be a set screw extending into the sleeve.

The engaging members may include a deformable element which engages a rod and secures the member to the rod when deformed. The deformable element may be a tulip fastener

The engaging members may include clamps for clamping rods.

The combined securing means may be a combined longitudinal and pivot securing means wherein the combined securing means includes a set screw which acts radially on the longitudinal rod such that it is engaged by the longitudinal engaging member and the rod simultaneously actuates the pivot securing means in order to pivotally secure the longitudinal and transverse members relative to each other.

The engaging members may be pivotally attached to each other through a spherical bearing such as a ball joint.

The engaging members may be pivotally attached to each other about a central pin, such that, operatively when the engaging members engage rods, the rods may pivot relative to each other about a common plane.

In accordance with a second aspect of the invention there is provided a system for spinal corrective surgery comprising:
two spinal fasteners including rod engaging means;
a transverse spinal rod and a longitudinal spinal rod;
the transverse spinal rod being securable to a vertebra through the two spinal fasteners which may be fastened to the vertebra end engaged to the transverse spinal rod;
a coupler including transverse and longitudinal rod engaging members which are pivotable relative to each other;
each rod engaging member including securing means for securing the engaging member to a rod and the coupler including pivot securing means for pivotally securing the engaging members relative to each other;
wherein, operatively, the transverse spinal rod may be affixed to a vertebra with the transverse rod engaging member of the coupler engaging the transverse spinal rod such that the coupler is movable longitudinally along the transverse spinal rod; the longitudinal engaging member may engage the longitudinal spinal rod such that the coupler is movable longitudinally along the longitudinal spinal rod; and the relative positions of the vertebra, the transverse spinal rod, the longitudinal spinal rod, and the coupler may be secured by the securing means.

The system may include a plurality of transverse spinal rods, each with an associated coupler, for securing the transverse spinal rods to a single longitudinal spinal rod.

The system may also include a plurality of longitudinal spinal rods.

The spinal rods may be curved.

### BRIEF DESCRIPTION OF THE DRAWINGS

An embodiment of the invention is described below, by way of example only, and with reference to the drawings in which:

FIG. 1 is a schematic perspective view of a spinal rod coupler and transverse rod;

FIG. 2 is a schematic front view of a spinal rod coupler and transverse rod;

FIG. 3 is a schematic perspective view of a spinal rod coupler;

FIG. 4 is a schematic transverse cross-sectional view of a vertebra showing an implanted transverse rod, a spinal rod coupler, and cross-sectional view of a longitudinal rod;

FIG. 4a is a schematic side of a transverse rod, a spinal rod coupler, and cross-sectional view of a longitudinal rod;

FIG. 5 is a schematic posterior view of a vertebra showing implanted transverse and longitudinal rods engaged by a spinal rod coupler

FIG. 5a is a schematic posterior view of transverse and longitudinal rods engaged by a spinal rod coupler

FIG. 6 is a schematic lateral view of a vertebra with implanted transverse and longitudinal rods engaged by a spinal rod coupler;

FIG. 6a is a schematic lateral view of transverse and longitudinal rods engaged by a spinal rod coupler;

FIG. 6b is a schematic lateral view of transverse and longitudinal rods engaged by a spinal rod coupler;

FIG. 7 is a schematic plan view of a pedicle screw;

FIG. 8 is a schematic lateral view of a spine with a system for spinal corrective surgery;

FIG. 9 is a schematic posterior view of a spine with a system for spinal corrective surgery;

FIG. 10 is a schematic exploded perspective view of a second embodiment of a spinal rod coupler; and

FIG. 11 is a schematic exploded perspective view of a third embodiment of a spinal rod coupler.

### DETAILED DESCRIPTION OF THE DRAWINGS

With reference to the drawings in which like features are indicated by like numerals, a spinal rod coupler is generally indicated by reference numeral 1.

The coupler 1 has a first rod engaging member 2 and a second rod engaging member 3 which are pivotally attached to one another such that the engaging members may pivot relative to each other. The engaging members are shaped and sized to engage spinal rods with the first engaging member 2 being adapted to engage a transverse spinal rod and the second engaging member adapted to engage a longitudinal spinal rod. For convenience, the first engaging member will be referred to as the transverse member and the second engaging member 3 will be referred to as the longitudinal member 3.

The coupler 1 is intended for use with a system for spinal corrective surgery wherein the system includes a plurality of transverse rods 4 which each rod extending transversely across the posterior of a vertebra. The transverse rods 4 are typically curved such that the concave side of the curvature faces the vertebra. The transverse rods are affixed to a vertebra by first mechanically removing portions of the vertebra which might interfere with the implantation thereof and affixing the rod 4 to each vertebra through two pedicle screws 6 which engage the rod 4.

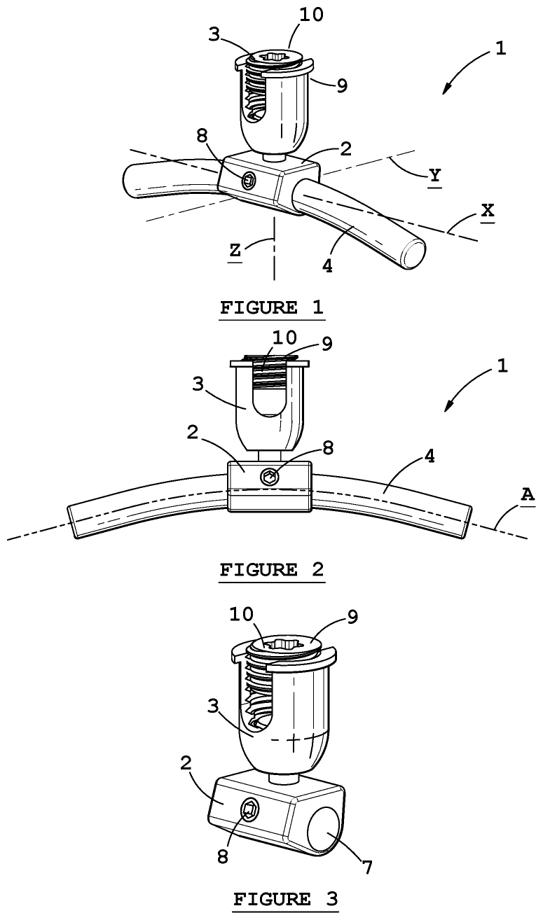

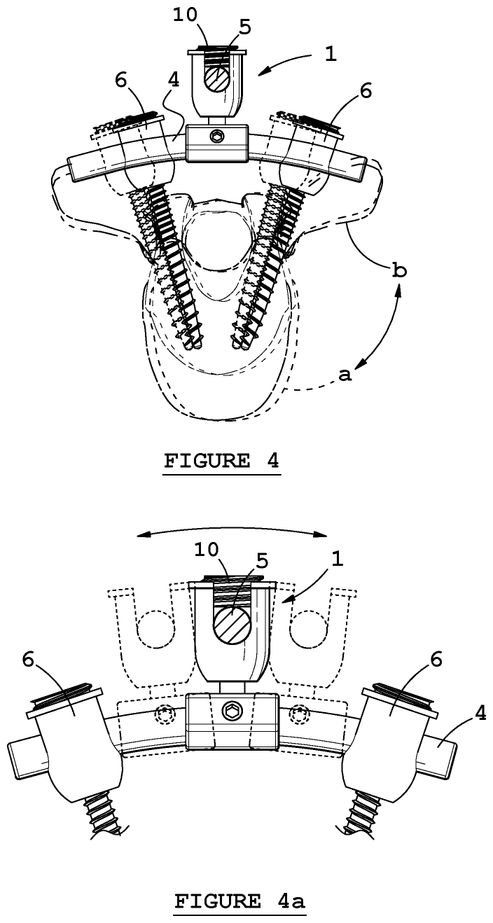

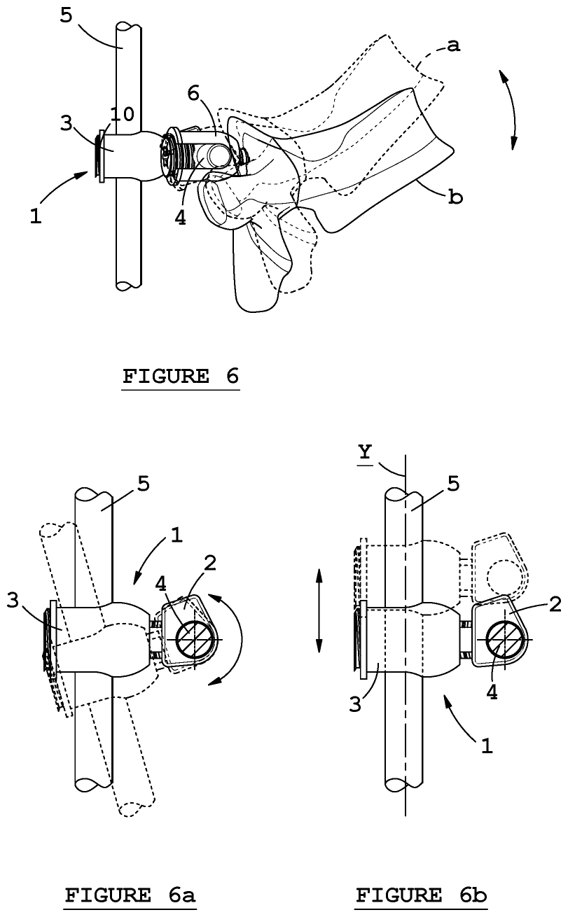

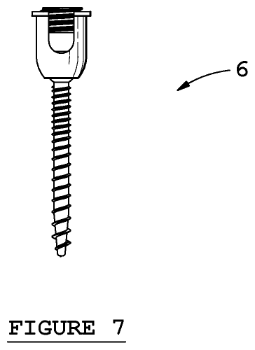

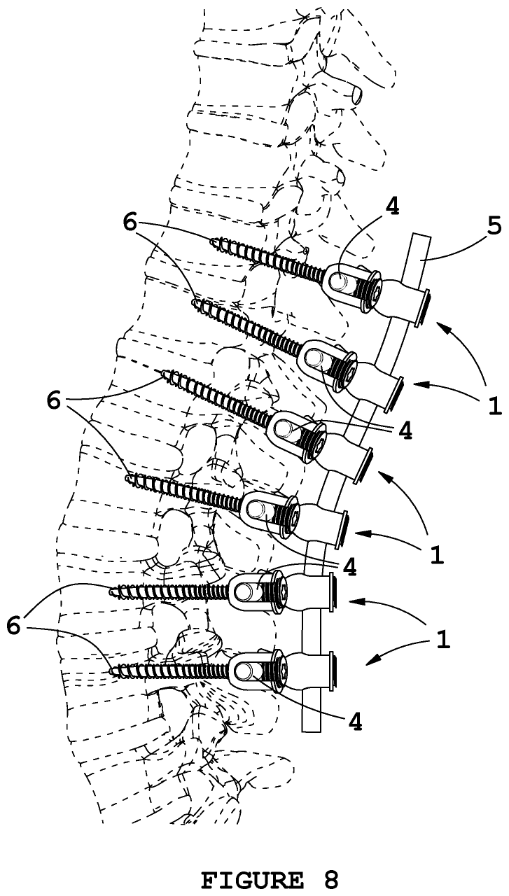

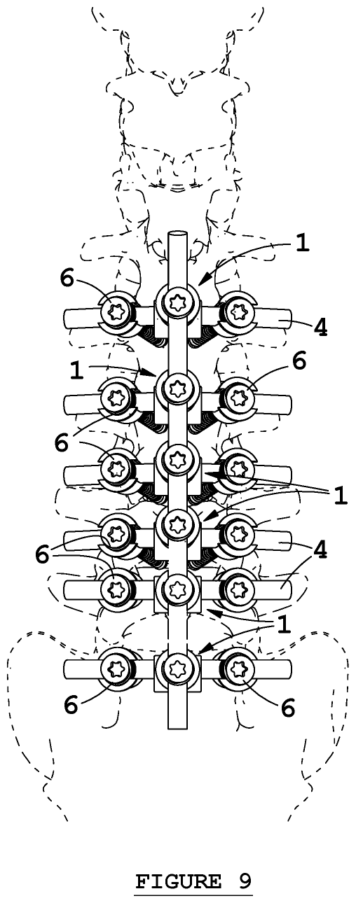

The transverse member 2 is engageable to a transverse rod 4 such that, when the transverse member 2 engages the transverse rod 4, the transverse member 2 is movable longitudinally along the length the transverse rod 4 (transverse relative to the patient as shown in FIG. 4a). In the embodiment shown in FIGS. 1 to 9, this is accomplished by a sleeve, or aperture, 7 shaped into the transverse member 2 which may be installed (threaded) onto a transverse rod 4 prior to affixing the rod 4 to a vertebra. The inner diameter of the sleeve 7 is slightly larger than the outer diameter of the rod 4 such that the sleeve 7, and consequently the coupler 1, is slidably movable along the rod 4. The transverse member 2 also includes securing means, shown in this example as a set screw 8 which extends into the sleeve 7. The set screw 8 allows position of the transverse member 2 to be secured to the rod 4 both longitudinally along the rod 4 (in other words the position along the length of the rod 4 as indicated by the bidirectional arrow and positions of the coupler in dashed lines in FIG. 4a) and also rotationally about the rod 4 (as shown in FIG. 6a).

The system also includes a longitudinal rod 5 which runs longitudinally along the length of the spine (or at least the portion of the spine wherein the system is implanted). The system described and depicted in this example has a single longitudinal rod 5 extending along the spine and the applicant submits that the single longitudinal rod 5 is a preferred embodiment as it decreases the size and profile of the system when implanted. However, the system may include a plurality of longitudinal rods or shorter sections of longitudinal rod where the use of multiple rods may be necessary.

The longitudinal member 3 engages the longitudinal rod 5 along the length of the longitudinal rod 5 in such a way that it is movable along the longitudinal rod 5 (as is shown in FIG. 6b and also includes securing means for securing the longitudinal member 3 to the longitudinal rod 5 in the form a set screw 10. Alternatively, the longitudinal member 3 may engage the rod 5 through a clamping mechanism whereby at least part of the member 3 is deformed (through external mechanical manipulation) to frictionally engage the rod 5. In the example described herein the longitudinal member 3 has an open side allowing a rod to be engaged without threading as described for the transverse member 2. The longitudinal member 3 also includes protrusions 9 which allow the longitudinal member 3 to be engaged by a tool which is used to manipulate the rod into the longitudinal member 3 for engagement. The longitudinal member 3 in this example will have at least have some purchase on the rod through mechanical deformation but may be further secured to the rod 5 by securing means in the form of set screw 10. The set screw may be tightened to secure the longitudinal member 3 along the length of the rod 5.

The coupler 1 includes pivot securing means for pivotally securing the transverse 2 and longitudinal 3 members relative to each other. This may be in the form of an additional set screw in the pivot of the coupler 1, or form part of combined securing means wherein the frictional engagement of the rod 5 to the longitudinal member 3 will also frictionally secure the pivot of the coupler 1. In this example, the combined securing means is in the form of combined longitudinal and pivot securing means wherein the combined securing means includes a set screw 10 which acts radially on the longitudinal rod 5 such that it is engaged by the longitudinal engaging member 3 by forcing and frictionally engaging the rod. This engagement between the rod 5 and the member 3 causes the rod 5 to simultaneously actuate the pivot securing means in order to pivotally secure the longitudinal and transverse members relative to each other. This is achieved by the rod 5 engaging the top of the pivot (which is visible in FIGS. 1,2, and 3) and thus cause relative motion between the longitudinal member 3 and the pivot. This relative movement will engage an internal portion of the pivot to an internal portion of the longitudinal member 3 which secures relative pivotal movement. In other words, the member 3 is locked onto the longitudinal rod 5 through means of a friction interface to restrain all motion on the rod 5 through set screw 10 imparting increased friction on the rod 5. In turn the tightening of the set screw 10 on the longitudinal rod 5 will impart increased friction on the rotational/pivotal mechanism which will lock any pivotal movement between the transverse 2 and longitudinal 3 members.

The member 3 as indicated in the figures has a shape similar to what is referred to in the industry as a screw tulip. At the time of writing, the applicant believes that a mechanism which is similar to a Stryker K2M Mesa′ screw head will be very beneficial to the invention. In addition to its relatively low profile, the Stryker K2M Mesa′ head has a clamp action onto the rod 5 which does not indent the rod 5 or cause deformation that might potentially weaken the rod 5. The coupler may include combined securing means wherein one or more of the first securing means, the second securing means, and the pivot securing means is actuated my a single mechanism

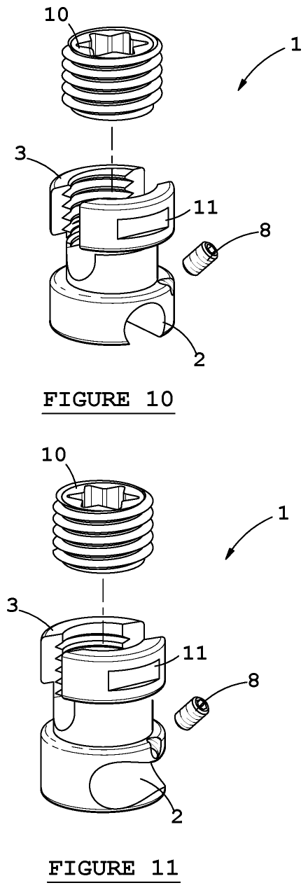

A second embodiment of the coupler 1 is shown in FIG. 10 and a third embodiment of the coupler 1 is shown in FIG. 11. In both embodiments, the coupler 1 has a substantially cylindrical body. The second embodiment has a transverse rod engaging member 2 with a central groove, roughly C-shaped and sized to engage a transverse rod 4 in a snap-fit manner or to thread the rod 4 through the groove, which extends centrally through the member 2. In the third embodiment, a similarly shaped channel tangentially along an outer periphery of the member 2. The second and third embodiments both have a grub screw 8 for securing a transverse rod 4 to the member 2.

Both the second and third embodiments have a threaded slot in the longitudinal member 3. The member has flats 11 in the member 3 for engaging the member 3 with a tool (such as an appropriately sized spanner or a wrench) in order to manipulate the member and the coupler 1. Both the second and third embodiments have a set screw 10, which is shaped and sized to engage the threaded portion of the member 3 in order to secure a longitudinal rod 5 therein.

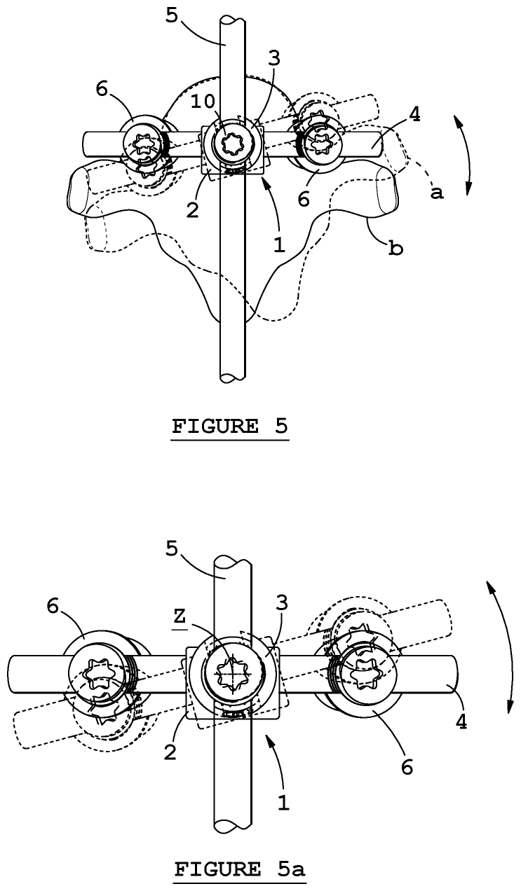

The members and securing means described above allows the position of the coupler 1 and the relative positions of rods engaged by the coupler 1 to be adjusted and secured with great flexibility. This provides a surgeon with many degrees of freedom for adjustment. In practice, a surgeon would manipulate the individual vertebra and then secure the coupler 1 to maintain the vertebra in the desired position. For example, as can be seen in FIG. 4, by adjusting the longitudinal position of the coupler 1 along the length of the curved transverse rod 4, the rotation of the vertebra about the axis of the spine (roughly as indicated by the bidirectional arrow in the figure) may be accurately adjusted (for example from position b to a and vice versa as shown in FIG. 4) before the position is secured. Similarly, as can be seen in FIG. 5, the rotation of a vertebra in the coronal plane may be accomplished by pivotally manipulating the relative pivotal position between the transverse rod 4 and longitudinal rod 6 and maintained by securing the pivotal securing means. Further, it is possible to secure the rotation of a vertebra in the sagittal plane by adjusting the longitudinal position of the longitudinal member 3 and the relative pivotal position between the members as shown in FIG. 6. The flexibility and multiple degrees of freedom available allow the surgeon to manipulate, rotate, and/or translate individual vertebrae and, by systematically securing the couplers, allows the surgeon to manipulate the patient's spine into a desired position.

It is envisaged that the invention will provide a spinal rod coupler and related system for spinal corrective surgery which provides surgeons with multiple degrees of freedom for manipulating a patient's vertebrae and spine and to secure these manipulations in an easy and effective manner.

The invention is not limited to the precise details as described herein. For example, instead of having a sleeve on the engaging members, integrated clamps may be used to engage and secure the rods.

1. A spinal rod coupler comprising: a first rod engaging member and a second rod engaging member pivotably attached to each other; the first engaging member being engagable to a first rod such that, when engaged, the member is movable longitudinally along the first rod and the first engaging member having first securing means for securing the engaging member to the first rod longitudinally along the first rod; the second engaging member being engagable to a second rod such that, when engaged, the member is movable longitudinally along the second rod and the second engaging member having second securing means for securing the second rod engaging member to the second rod longitudinally along the second rod; and a pivot securing means for pivotally securing the first rod engaging member and second rod engaging member relative to each other; wherein a position of the coupler and relative positions of the rods is adjusted longitudinally along the rods and pivotally between the rods and secured in place by the securing means.

2. The coupler according to claim 1, further comprising a combined securing means wherein one or more of the first securing means, the second securing means, and the pivot securing means is actuated by a single mechanism.

3. The coupler according to claim 1 or 2, wherein the engaging members are spinal rod engaging members.

4. The coupler according to claims 1 to 3, wherein the first rod engaging member is a transverse rod engaging member for engaging a transverse rod and the second rod engaging member is a longitudinal rod engaging member for engaging a longitudinal rod which extends crosswise, or at any angle, relative to the transverse rod.

5. The coupler according to claims 1 to 4, wherein the rod engaging member includes a sleeve which slidably engages a rod, wherein the sleeve is an enclosed tubular structure or an open channel to accommodate the rod or member.

6. The coupler according to claim 5, wherein the channel is U-shaped.

7. The coupler according to claim 5, wherein the channel is C-shaped channel.

8. The coupler according to claim 5, wherein the sleeve includes securing means in the form of a fastener for fastening the sleeve to a rod,

9. The coupler according to claim 8, wherein the fastener is a set screw extending into the sleeve.

10. The coupler according to claims 1 to 9, wherein the engaging members include a deformable element which engages a rod and secures the member to the rod when deformed.

11. The coupler according to claim 10, wherein the deformable element is a tulip fastener.

12. The coupler according to claims 1 to 11, wherein the engaging members include clamps for clamping rods.

13. The coupler according to claim 2, comprising the combined securing means is a combined longitudinal and pivot securing means wherein the combined securing means includes a set screw which acts radially on the longitudinal rod such that it is engaged by the longitudinal engaging member and the rod simultaneously actuates the pivot securing means in order to pivotally secure the longitudinal and transverse members relative to each other.

14. The coupler according to claim 1 or 2, wherein the engaging members are pivotally attached to each other through a spherical bearing such as a ball joint.

15. The coupler according to claim 1 or 2, wherein the engaging members are pivotally attached to each other about a central pin, such that, operatively when the engaging members engage rods, the rods pivot relative to each other about a common plane.

16. A system for spinal corrective surgery comprising: two spinal fasteners including rod engaging means; a transverse spinal rod and a longitudinal spinal rod; the transverse spinal rod being securable to a vertebra through the two spinal fasteners which are fastened to the vertebra end engaged to the transverse spinal rod; a coupler including transverse and longitudinal rod engaging members which are pivotable relative to each other; each rod engaging member including securing means for securing the engaging member to a rod and the coupler including pivot securing means for pivotally securing the engaging members relative to each other; wherein, operatively, the transverse spinal rod is affixed to a vertebra with the transverse rod engaging member of the coupler engaging the transverse spinal rod such that the coupler is movable longitudinally along the transverse spinal rod; the longitudinal engaging member engages the longitudinal spinal rod such that the coupler is movable longitudinally along the longitudinal spinal rod; and the relative positions of the vertebra, the transverse spinal rod, the longitudinal spinal rod, and the coupler are secured by the securing means.

17. The system according to claim 16, comprising a plurality of transverse spinal rods, each with an associated coupler, for securing the transverse spinal rods to a single longitudinal spinal rod.

18. The system according to claim 16, includes a plurality of longitudinal spinal rods.

19. The system according to claim 16, wherein the spinal rods are curved.
